(19) United States
(12) Patent Application Publication
(10) Pub. No.: US 2023/0345943 A1
(43) Pub. Date: Nov. 2, 2023

(54) METHOD AND PROCESS FOR COMPLETE ELIMINATION OF VARIOUS STEPS OF PEST GROWTH IN PLANTS USING DISINFECTANT COMPOUNDS BASED ON ETHANEPEROXOIC ACID AND HYDROGEN PEROXIDE

(71) Applicant: Sasan Sartipi, Tehran (IR)

(72) Inventor: Sasan Sartipi, Tehran (IR)

(21) Appl. No.: 18/025,898

(22) PCT Filed: Sep. 19, 2020
(86) PCT No.: PCT/IB2020/058753

§ 371 Date: Mar. 13, 2023

(51) Int. Cl. A01N 59/00; A01N 37/02; A01P 7/04; A01N 25/32; A01P 1/00

(52) U.S. Cl. CPC A01N 59/00; A01N 37/02; A01P 7/04; A01N 25/32; A01P 1/00

(57) ABSTRACT

The invention of process and method for complete elimination of various stages of pest growth in plants using disinfectant compounds based on ethaneperoxoic acid and hydrogen peroxide which is related to a method to control and eradicate pests and fungal diseases and bacteria and viruses in plants, with the ability to have a simultaneous effect on both pests and plant diseases without creating resistance, by dissolving the ethaneperoxoic acid with a water solvent and fatty amin as wetting agent. The mechanism of function of ethaneperoxoic acid is oxidation; The oxidation mechanism involves electron transfer, ethaneperoxoic acid breaks sulfhydryl (H—S) and sulfide (S—S) bonds in proteins and enzymes and destroys the cell wall and thus oxidizes the outer membrane of bacterial cells, endospores, yeasts and mold spores, thus do the disinfecting process. This means that this disinfectant, in addition to killing many microorganisms, can also kill spores.

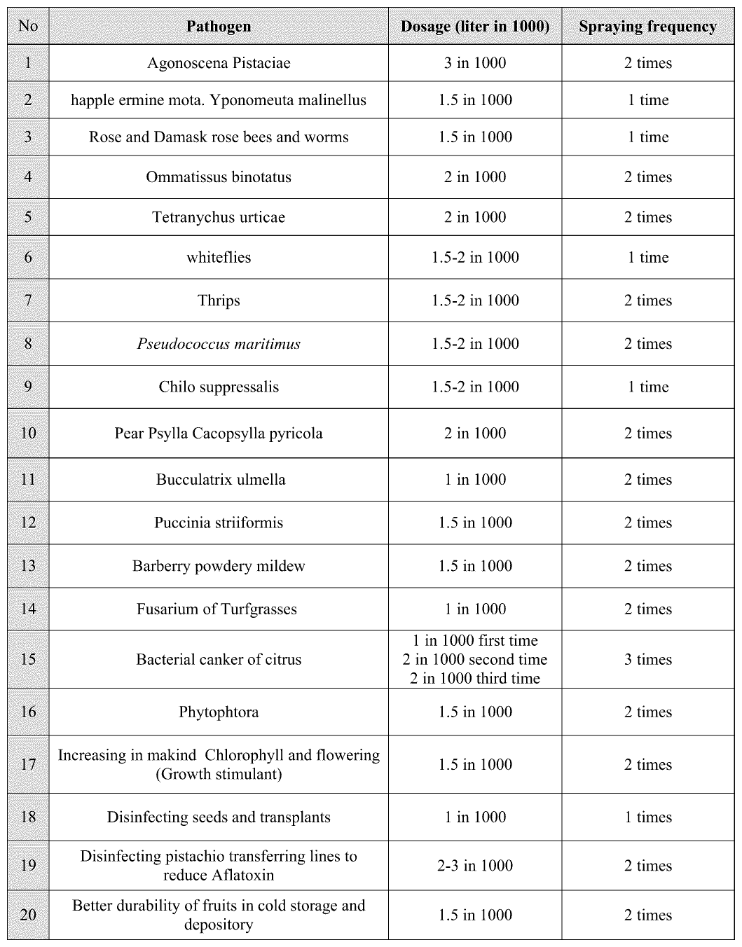

### TECHNICAL FIELD OF THE INVENTION

The present invention relates to a method to control and eliminate pests and fungal disease and bacteria and viruses in plant and also a method to prevent from plants resistance against chemical pesticides and also a method to disinfect the plant's pests.

### TECHNICAL ISSUE AND PURPOSE OF THE INVENTION

During current years, the world's population is increasing with a growing speed and is reaching to eight billion people, therefore to provide the essential and required food for this population, the manufacturers of agricultural products, apply more chemical pesticides to produce more products.

These chemical pesticides create some problems such as environmental pollution, surface water and ground water pollution, chemical pesticides residual in environment, pest resistance to these chemical pesticides and endangering food security and safety; in addition, these residue have adverse effects on human health like irrevocable genetic mutation, carcinogenesis effects and mutagen effects on natural organs of the body.

On the other hand, human beings by having the knowledge of the dangers of excessive use of chemical pesticides, have moved towards organic farming, which one of the problems of organic cultivation is low function and therefore less agricultural production, also, production costs of organic farming are much expensive and not cost-effective. Therefore, the academic community and inventors have achieved the important point to use compounds with non-chemical, non-toxic, environmental and eco-friendly properties, without residues in agricultural and biodegradable products and for this reason, in recent years, the use of herbal compounds and extracts has become common in South America and the European Union which is again not cost effective in comparison with chemical pesticides.

According to above, it is required to first use eco-friendly and biodegradable compounds without pre harvest interval, second do not have any residue and should have appropriate effectiveness on plant pathogens and doesn't weak as like as herbal extracts and be highly economical. Hence, researches has been done on biocide compounds which are based on Ethaneperoxoic acid and according to this, it will immediately convert to acetic acid and hydrogen peroxide once it spatters and gets in to contact with pests on plant tissues in the farm.

Following to the usage of pesticides human unintentionally, accidentally or inevitably face with these pesticides and its remain through air, water, earth, plants, animals and inanimate objects that can have adverse effects on human health including carcinogenicity and genetic mutations. Also, today there is no pesticide that is completely safe for the environment and safe for human health. Another problem that should mention is the fact that overuse of agricultural pesticides actually causes many pests to become resistant to the pesticides. On the other hand, these toxins remain in the environment after use. Given to creating resistance in a wide range of pests and the ineffectiveness of various pesticides and the resulting problems, it is noteworthy that compounds based on ethaneperoxoic acid can eradicate plant pests and diseases without creating resistance in them.

Most pests after several periods of frequent use from pesticides, become resistance to them which this resistance has different types.

### 1. Reduced Penetration of Pesticides

In this type of resistance, some changes occur on chitin shell of insects which prevent from penetrating the pesticide inside the body.

### 2. Resistance Through Breaking the Pesticide's Formulation by Insect's Enzymes (Metabolic Resistance)

In this type of resistance, the enzymes of insect's body and the enzymes which increase when pesticide gets in to contact with the insect's body, slightly and qualitatively change which break down the pesticide and neutralize their effects.

### 3. Changes in Target Systems of Insects (Target Site Insensitivity)

The mechanism of action of pesticides on insects is mostly on their nervous system. In this type of resistance which is one of the worst resistance mechanisms in insects, the insect cause the pesticides to become ineffectiveness by making some changes in the target structure of pesticides which are almost ion channels. For instance, the insect by changing in the number of sodium and potassium ion channels and their reduction cause the resistance. In addition, the chemical changes in insect's structural and molecular units reduce the affinity of pesticides in the target site and make resistance. Having resistance to phosphorus pesticides is due to structural changes in the enzyme of Acetyl cholinesterase and therefore the pesticide doesn't adhere to the enzyme and ultimately will not cause the transmission of neural messages.

### 4. Behavioral Changes

Due to the stimulus effects of pesticides on pests, the insects avoid contact with the pesticides or do not enter to places where it was sprayed by pesticides. This type of resistance mechanism is actually the result of physiological changes in the body. According to these facts, a compound without the pesticides chemical specifications have to be used and in frequent use of these Ethaneperoxoic acid based compounds, no resistance is reported against this type of compound given to different resistance mechanisms.

Now, copper compounds are used to fight against some fungi and bacteria which in addition to being harmful to the environment, copper compounds cause poisoning in high doses and lead to environmental pollution.

Therefore, the purpose of present invention is to use a method to fight against wide range of pests based on ethaneperoxoic acid which can be effective without remaining in environment (residue) and also it is non-toxic and eco-friendly, which for the result of its use can mention to preventing types of cancers and minatory factors for animals and human health.

### PRIOR ARTS

From past, the ethaneperoxoic acid is used as a disinfectant to reduce the level of pollution and eradicate bacteria, fungi and virus. The compounds based on ethaneperoxoic acid are effective on wide range of fungus:
1. Pythium
2. Fusarium
3. Aspergillus
4. Phytophtera

And also it is effective on wide range of bacteria's such as Bacillus sp, Mycobacterium sp, Clostridium sp, coliform, Escherichia coli, Staphylococcus aureus, Xanthomonas axonopodis pv. Citri.

Various chemical pesticides have been used to control pests: 10% diazinon granules in amount of 15 kg per hectare, 5% diazinon granules in amount of 30 kg per hectare and Padan 4% (kartap) in amount of 25 kg per hectare.

A patent with DE3902009A1 patent number filed in Germany's Patent Office named “Method of controlling pests on plants” is a way to control pests on plants and arable crops using an aqueous solution containing hydrogen peroxide (H2O2) or ozone in the form of a spray that has no effect on environmental pollution. If commercial hydrogen peroxide at a concentration of 35% used, the concentration of hydrogen peroxide in the aqueous solution that acts as spray will preferably remain in the range of about 1.5% and reactive oxygen species form a group consist of ethaneperoxoic acid, hydrogen peroxide, calcium peroxide, potassium percarbonate, sodium percarbonate and urea peroxide.

A patent with WO1999044419A1 patent number filed in WIPO named “Methods for control of insects on plants, control of horticultural diseases, and decontamination of plant tissue” is a method of controlling insects on plants, gardening diseases and plant tissue contamination, which includes the use of a solution containing 0.05 to 3.00% hydrogen peroxide, which is a spray solution that is applied to the roots of trees and contains acids such as acetic acid or phosphoric acid and the solution is applied with hydraulic spray.

### DESCRIPTION OF THE INVENTION

In the presented method of this invention, for the first time, compounds based on ethaneperoxoic acid were used to control various agricultural and horticultural pests, which with this method, a disinfectant is used to eradicate different developmental stages (eggs, pupa, larvae and full insect).

According to the method of this invention, dissolving ethaneperoxoic acid with water solvent leads to the production of hydrogen peroxide and acetic acid, which by producing billions of free radicals, can controls and prevents a variety of fungi, viruses, bacteria and pests. In fact, ethaneperoxoic acid is a potential oxidizing agent consisting of hydrogen peroxide and acetic acid that dissolves easily in water and decomposes into non-toxic and harmless by-products like water, acetic acid and oxygen and this product can be introduced as the best oxidizer after ozone. In addition, these compounds remain no residue in the environment and are completely ecofriendly. The mechanism of function of ethaneperoxoic acid is oxidation; The oxidation mechanism involves electron transfer, ethaneperoxoic acid breaks sulfhydryl (H—S) and sulfide (S—S) bonds in proteins and enzymes and destroys the cell wall and thus oxidizes the outer membrane of bacterial cells, endospores, yeasts and mold spores, thus do the disinfecting process. This means that this disinfectant, in addition to killing many microorganisms, can also kill spores.

These compounds are in the GRAS list (generally recognized as safe), as a result have significant effects on herbal pathogens, viruses, fungus and chitin coverage of pests.

Using these compounds in the appropriate dose has no negative effect on plant tissue such as leaf burning and leaf fall, etc. It should be noted that for potential impact and increase the function of ethaneperoxoic acid (with appropriate dose), surface tension reducing compounds must be used (wetting agent).

The mechanism of compounds effectiveness based on Ethaneperoxoic acid is according to oxidation which contains the transfer of electrons. The Ethaneperoxoic acid breaks the sulfhydryl (H—S) and sulfide bonds in proteins and enzymes and destroys the cell walls, oxidize the outer membrane of pests chitin cell walls and bacterial cells, endospores, yeasts and mold spores and therefore eradicates the plant pathogens. This means that this disinfectant besides eradicating many micro organisms, can also eradicate spores.

Due to the fact that the mechanism of compounds which are based on Ethaneperoxoic acid is by contact (touch) and not create toxicity on cells, so far no resistance due to their use has been reported and as a result the compounds with these characteristic will make a major changes in the agricultural industry.

Another characteristic of Ethaneperoxoic acid based compounds which were found during the field tests, in addition to acting on pests and herbal pathogens, strongly leads to succulence, growth accession, increasing vegetation mass and formation of chlorophyll which seems that the stimulant of plant for increasing its growth and blooming as well as increasing the vegetation mass is because the plant is in tangle with harmful pathogens like bacteria, virus and saprophyte fungus in its surroundings which based on the amount of these pathogens, the plants spend part of the energy that is for activation of immune system to fight with these pathogens. When the Ethaneperoxoic acid based compounds are used, the plants were succulence, the blooming and the production increased which seems that population accession and load of pathogens reduced using biocide compounds and ultimately leads to the elimination of these factors. As a result, the plant spends the energy of fighting harmful plant pathogens for increasing the production and generally the plant tissues.

For longer durability of Ethaneperoxoic acid based compounds, non-ionic fatty amine compounds (wetting agent) should be used in this method for the first time in two different ways, both within the formulation or added separately to the compound. These compounds cause the Ethaneperoxoic acid that is sprayed on the target to increase the duration of exposure of Ethaneperoxoic acid to the chitin surface of insects due to the reduction of surface tension of water droplets. The use of 500 cc of non-ionic fatty amine compounds and 3 liters of Ethaneperoxoic acid (with an active ingredient of at least 20%) in 1000 liters of water, can have a significant impressive effect in controlling fungus, bacteria and viruses and plant pests in all stages such as eggs, larva, adults, etc.

### DESCRIPTION OF DRAWINGS

In order to control a wide range of plant pests and diseases, a specific dose of ethaneperoxoic acid was used, which is shown in Table 1.

### Advantages of the Invention

No creation of resistance toward ethaneperoxoic acid due to rapid impact
Economical
No need for fungicides or bactericides to kill fungi and other pathogens
Improvements in the quality and quantity of the final product
ethaneperoxoic acid is widely produced in various concentrations and by creating a market for ethaneperoxoic acid to fight with a variety of pathogens (fungi, bacteria and viruse) as well as fighting with plant pests, naturally, it creates more product and farm yields qualitatively and quantitatively.
Has no negative effect on plant tissue such as leaf burn and leaf fall, etc. in appropriate dosage with fatty amin (wetting agent)

### DESCRIPTION OF A PRACTICAL SAMPLE

The Chilo suppressalis

This worm is one of the important pests of rice paddy of Gilan, Mazandaran and Gorgan. This pest lives as a larva in dried rice haulm or weeds with hallow haulm such as Coix lacryma-jobi, Cockspur grass, Perennial sow thistle, Phragmites australis, Sambucus nigra, Millet, Musk, Cynodon dactylon whole the winter. The first pupas come from winter's larva, form in last 10 days of April next to the hole made by the larva. This pupa is without cocoon. The minimum temperature to become pupa is 10° C.; the pupa's period of life has reverse relationship and change from 15 to 30° C. between 6 to 12 days. The pupas damage higher than 35° C. The more weight of pupas, the more oviparity by female butterflies, come out of it. In Mazandaran, the first butterflies exit in late April. The male and female butterflies copulate typically 24 hours after coming out of pupa and their lifetime is at most a week. The first generation butterflies (wintry) generally lay their eggs above or under the upper leaves, however, the second generation butterflies almost lay their eggs on inferior leaves or on the haulm and near the leaves. Breeding of eggs should be done in at least 10-12° C. in 5-11 days based on the outside temperature.

After breeding, the first generation larva first feed from leaf's Parenchyma and then enter to haulm by creating a hole from petiole. The larva become pupa after 21 to 37 days and come out from pupa 7 days later the second generation (spring) butterflies, the maximum number of this generation are from late July. Chilo suppressalis has 3 generations in north of Iran. The first generation is associated with transplantation. Therefore, the damage of this generation is related to the fields that were transplanted earlier. The second generation of the pest is when rice is clustering and many of them are not in danger of severe damage. The third generation coincides with the rice harvest and will cause significant damage to late-ripening, mid-clay and ratoon rice. The larva is amazingly resistant to water logging and winter cold, but is highly sensitive to drought and temperature changes and suffers severe mortality. The first generation begins from mid April and continues to early July, the second generation from early July to late August and the third generation from late august to late October. This pest damage the unripe and mid-clay rice in at most two generations and in late-ripening rice, damage the three generations. The third generation larva remain in stubbles due to the air cooling and rice harvesting and the weeds at the side of the farm, stacks and stubble's piles are also remain till the next spring. Typically in ideal conditions, each generation of Chilo suppressalis continues among 45 to 50 days.

In this invention ethaneperoxoic acid used for the first time to fight with Chilo suppressalis with the below mentioned method:
Step 1: The dosage of 1 in one thousand in the transplantation treasury;
Step 2: At the time of transferring the seedlings to the field with the dosage of 1 in one thousand to fight with the first generation;
Step 3: 10 days after transferring the seedlings to the field with the dosage of 1 in one thousand to fight with the second generation;
Step 4: 30 days after transferring the seedlings to the field with the dosage of 1 in one thousand;
Step 5: 45 days after transferring the seedlings to the field with the dosage of 2 in one thousand and 1 in one thousand to fight with the third generation;
Step 6: before harvesting the rice with the dosage of 2 in one thousand.

By doing the above protocol, the damage due to the Chilo suppressalis would completely control.

### The Agonoscena Pistaciae

Agonoscena Pistaciae is known as dry sap in some areas of pistachio fields, because the excreted honeydew resulting by this pest quickly loses its moisture and remains as a white or milky crystals on the tree's bough, stick and leaf. The density of this pest in some gardens is so severe that the entire surface of the sticks and boughs of trees and the shady ground of the tree is covered with honeydew. Pistacia terebinthus and domestic pistachio trees are the hosts of this pest and its severe damage has been reported from almost all pistachio-growing areas of Iran and countries in the region.

The damage of this pest is due to excessive sucking of plant sap, which causes severe weakness of the tree, small fruit fall, remaining fruits being hollow and in high density leads to leaf fall.

Agonoscena Pistaciae spends the winter in the form of full insects, under dry leaves, tree flake and on pistachio twigs. It seems that its resistance to adverse weather conditions, especially temperature changes in winter, is more or less similar to pear psyllids and is well resistant to winter cold. In Kerman, adult insects appear in mid March. The oviparity starts very soon, once the weather warms up, even in March, and it lays eggs on the twigs and leaves. The larvas come out of eggs coincides with flourishing the buds and appearance of leaves. The larva excretes much honeydew which sometimes they themselves drown in their secretions. Thus, clusters, leaves, sticks, boughs and the ground which is under the shade of the tree, are heavily contaminated with honey. The nymphal period in the first generation is about 50 days, but by the weather warms, this period is reduced to 25-30 days. Pistachio psyllid is a multi-generation insect that can produce up to 5 generations per year depending on weather conditions.

### Previous Fighting Method:

1. Amitrase Mitak EC20% and 1/1.5 in one thousand=it is forbidden
2. Endosulfan EC35% and 1.5 in one thousand=it is forbidden

Since psyllids become resistant to toxin after using one type of it several times, try to use different toxins alternately.

Muspilan (acetamiprid), psyllids in early and late spring can be used in amount of half per thousand, Dartone (Phosalone+teflubenzuron), confidor (imidacloprid), with the dose of half in a thousand, Envidor, recommended dose Envidor 300 to 400 Cc per 1000 liters of water. For Consult (hexaflumorone), the recommended dose of hexaflumorone is half a liter per 1000 liters of water. The calypso (thiaclopride), Actara (Thiamethoxam) and Chlorpyrifos is recommended with the dose of 1.5 in one thousand to control the psyllids.

Recommendation: You can use 1 liter of Pirifus chlorine (Dorsban) and 0.5 liters of hexaflumorone (consult) plus 2 liters of soap in 1000 liters of water.

Anti-psyllid protocol using compounds based on ethaneperoxoic acid:
Step 1: The May to fight with the first generation, a dose of 1.5-2 in one thousand
Step 2: June to fight with the second generation, a dose of 3 in one thousand with surface tension reducing compounds at a dose of half in one thousand
Step 3: until September at intervals of once every 20 days at a dose of 3 in one thousand with surface tension reducing compounds at a dose of half in one thousand (July due to extreme heat interference of the second, third and fourth generations)

### EXPLICIT APPLICATION OF THE INVENTION

Usage of these compounds to control and fight with wide range of pests in agriculture.

What is claimed is:

1. The invention of method and process for complete elimination of various stages of pest growth in plants using disinfectant compounds based on ethaneperoxoic acid and hydrogen peroxide with the ability to have a simultaneous effect on both pests and plant diseases without creating resistance in them through dissolving ethaneperoxoic acid with water solvent together with fatty amin.

2. The invention of claim one in which hydrogen peroxide and ethaneperoxoic acid creates due to dissolving ethaneperoxoic acid with water solvent and results in producing free radicals to control and prevent different types of fungies, viruses, bacteria and pests.

3. The invention of claim one in which as soon as it release in the environment, leads to produce acetic acid, Oxygen and water.

4. The invention of claim one which this compound destroys the cell wall and, as a result, oxidizes the outer membrane of bacterial cells, endospores, yeasts, and mold spores, thus disinfecting is done.

5. The invention of claim one which these compounds leave no residue in the environment and are non-toxic.

6. The invention of claim one which is further to it function on pests and herbal pathogens strongly leads to succulence, growth accession, increasing vegetation mass and formation of chlorophyll.

7. The invention of claim one which the population accession and load of pathogens reduced using biocide compounds and ultimately leads to the elimination of these factors, as a result, the plant spends the energy of fighting harmful plant pathogens for increasing the production and generally the plant tissues.
